(19) United States
(12) Patent Application Publication
(10) Pub. No.: US 2007/0254956 A1
(43) Pub. Date: Nov. 1, 2007

(54) Medicament for Therapeutic and/or Preventive Treatment of Restenosis or Reocclusion After Vascular Recanalization Operation

(76) Inventors: Koichi Shudo, Tokyo (JP); Akiko Itai, Tokyo (JP); Susumu Muto, Tokyo (JP)

Correspondence Address: GREENBLUM & BERNSTEIN, P.L.C., 1950 ROLAND CLARKE PLACE, RESTON, VA, 20191, US

(21) Appl. No.: 10/577,487

(22) PCT Filed: Oct. 28, 2004
(86) PCT No.: PCT/JP04/16363

§ 371 Date: Mar. 14, 2007

(30) Foreign Application Priority Data
Oct. 29, 2003 (JP) 2003-368350

(51) Int. Cl. A61K 31/167; A61M 29/02; A61P 7/02; A61P 9/10

(52) U.S. Cl. 514/563

(57) ABSTRACT

A medicament for therapeutic and/or preventive treatment of restenosis or reocclusion after a vascular recanalization operation, which comprises as an active ingredient a compound represented by the following formula or a physiologically acceptable salt thereof, and a medical device for an intravascular treatment, which contains said compound or the physiologically acceptable salt thereof in a releasable form.

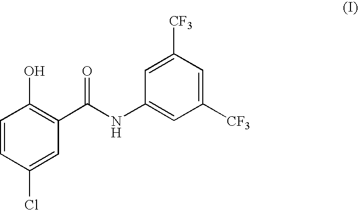

### FIELD OF THE INVENTION

The present invention relates to a medicament for therapeutic and/or preventive treatment of restenosis or reocclusion after vascular recanalization operation, and an intravascular indwelling medical device which enables prevention of said restenosis or reocclusion.

### BACKGROUND ART

Vascular recanalization operation mainly including intravascular intervention therapy using a catheter has become an important therapy for vascular occlusive diseases such as angina pectoris, and the number of the operation cases has rapidly increased, with improvements of catheters and standardization of maneuvers, as the operation is an epochal therapy for coronary artery diseases, peripheral artery diseases, furthermore, aortic and cerebral artery diseases. However, postoperative restenosis of the blood vessel in the lesion occurs at a frequency of as high as 40%, and therefore, prevention of the restenosis and post therapeutic treatment thereof have been problems. Recently, use of a stent has been introduced in many clinical cases after vascular recanalization operation, and occurrence ratio of restenosis has become lower than before. However, restenosis still occurs at a frequency of nearly 25%, and duration of recanalization effect has also been a problem. As a result of pathological researches so far made, restenosis is considered to be caused by an early thrombogenesis followed by proliferation of neointima. Although various studies have been made for achieving a therapy to inhibit proliferation of neointima, no effective therapy has been established so far.

Recently, it has been found that an activation of NF-κ B (Nuclear factor-kappa B) is significantly involved in the proliferation of neointima, and that NF-κ B oligo decoy, which is a double-stranded DNA fragment including NF-κ B binding sequences and has inhibitory activity against NF-κ B, strongly inhibits the proliferation of neointima in blood vessels after vascular recanalization operation. As a result, it has been revealed that NF-κ B inhibitors can be effective medicaments for therapeutic treatment of restenosis after vascular recanalization operation (Gene Therapy, (England), 2003, Vol. 10, pp. 356-364; Gene Therapy, (England), Vol. 8, pp. 1635-1642). The NF-κ B oligo decoy can be expected to have an actual preventive effect on restenosis after introduction of a stent in the human. However, the substance is a double-stranded oligomer consisting of a nucleic acid having about 20 base residues, and therefore, the substance is not a satisfactory optimum substance from a viewpoint of convenient handling of a medicament. Furthermore, from a viewpoint of synthetic costs, since the substance is difficult to be purified, high costs are still required at present to supply a bulk product which has a satisfactory purity as a medicament. Therefore, it is desired to develop therapeutic methods using a low molecular weight NF-κ B inhibitor which is lower-priced and convenient for handling.

As low molecular weight compounds having inhibitory action against NF-κ B, which are low-priced and convenient for handling, N-substituted salicylamide derivatives as prior art compounds are disclosed (the pamphlet of International Publication WO 02/49632). However, the publication fails to teach nor suggest whether or not the N-substituted salicylamide derivatives are useful for therapeutic and/or preventive treatment of restenosis caused by the introduction of a stent after a vascular recanalization operation. Furthermore, the publication also fails to teach nor suggest that the restenosis caused by the introduction of a stent can be prevented by applying said derivatives on an intravascular indwelling medical device such as a stent.

### DISCLOSURE OF THE INVENTION

An object of the present invention is to provide medicaments for therapeutic and/or preventive treatment of restenosis or reocclusion after a vascular recanalization operation, and an intravascular indwelling medical device which enables prevention of said restenosis.

The inventors of the present invention conducted various researches to achieve the aforementioned object, and as a result, they found that a medicament which comprises as an active ingredient a compound represented by the following formula or a physiologically acceptable salt thereof successfully exhibited extremely high effectiveness for therapeutic and/or preventive treatment of restenosis or reocclusion after a vascular recanalization operation, thereby the present invention was achieved.

The present invention thus provides a medicament for therapeutic and/or preventive treatment of restenosis or reocclusion after a vascular recanalization operation, which comprises as an active ingredient a compound represented by the following formula or a physiologically acceptable salt thereof.

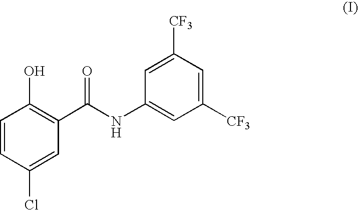

From another aspect, the present invention provides a method for therapeutic and/or preventive treatment of restenosis or reocclusion after a vascular recanalization operation, which comprises the step of administering a therapeutically and/or preventively effective amount of the compound represented by the aforementioned formula (I) or the physiologically acceptable salt thereof to a mammal including a human. According to preferred embodiments of the present invention, provided are the aforementioned method wherein the administration is carried out by a medical device for intravascular treatment which contains the compound represented by the aforementioned formula (I) or the physiologically acceptable salt thereof in a releasable form.

From further another aspect, the present invention provides a medical device, which contains the compound represented by the aforementioned formula (I) or the physiologically acceptable salt thereof in a releasable form. According to preferred embodiments of the present invention, provided are the aforementioned medical device, which is an intravascular indwelling stent or an intravascular balloon.

### BEST MODE FOR CARRYING OUT THE INVENTION

The compound represented by the aforementioned formula (I) or the physiologically acceptable salt thereof is disclosed in the pamphlet of International Publication WO 02/49632. Therefore, those skilled in the art can easily prepare the compound or the salt thereof. Kinds of salts are not particularly limited, and examples include metal salts such as lithium salt, sodium salt, potassium salt, magnesium salt, and calcium salt, or ammonium salts such as ammonium salt, methylammonium salt, dimethylammonium salt, trimethylammonium salt, and dicyclohexylammonium salt. As an active ingredients of the medicaments of the present invention, the compound represented by the aforementioned formula (I) or the physiologically acceptable salt thereof, or a hydrate thereof or a solvate may be used.

The medicament of the present invention can be used as a medicament for therapeutic and/or preventive treatment of restenosis or reocclusion after a vascular recanalization operation. Examples of the vascular recanalization operation include percutaneous transluminal coronary angioplasty using an intravascular stent or an intravascular balloon catheter. As the active ingredient of the medicament of the present invention, one or more kinds of substances selected from the group consisting of the aforementioned compound and a physiologically acceptable salt thereof, and a hydrate thereof and a solvate thereof may be used. As the medicament of the present invention, the aforementioned substance, per se, may be administered. Preferably, the medicament may be administered as a pharmaceutical composition for oral or parenteral administration which may be prepared by methods well known to those skilled in the art. Examples of the pharmaceutical compositions suitable for oral administration include tablets, capsules, powders, subtilized granules, granules, solution, and syrup, and examples of the pharmaceutical compositions suitable for parenteral administration include injections, infusions, suppositories, inhalants, percutaneous absorbents, and transmucosal absorptions.

The aforementioned pharmaceutical compositions can be prepared by addition of pharmacologically and pharmaceutically acceptable additives. Examples of the pharmacologically and pharmaceutically acceptable additives include excipients, disintegrators or disintegrating aids, binders, lubricants, coating agents, colorants, diluents, base materials, dissolving agents or solubilizers, isotonic agents, pH modifiers, stabilizers, propellants, and adhesives. A dose of the medicament of the present invention is not particularly limited. The dose may be appropriately increased or decreased depending on various factors that should be generally considered such as the weight and age of a patient, a kind and symptom of a disease, and an administration route, as well as the purpose of preventive or therapeutic treatment. Generally, for an oral administration, the medicament may be used in a range of 0.01 to 1,000 mg for an adult per day.

The medical device provided by the present invention is that used for an intravascular treatment in a vascular recanalization operation, and examples include intravascular stents or intravascular balloon catheters. Examples also include surgical devices such as artificial blood vessels, medical tubes, and medical clips, artificial valves, a part or the whole of artificial hearts, or endoscopes. The materials of the medical devices are not particularly limited. Any material that are usually used for manufacturing medical devices may be used, and examples include, metals, plastics, polymers, biodegradable plastics, biodegradable polymers, proteins, cellulose, and ceramics. The medical device of the present invention contains the compound represented by the formula (I) or the physiologically acceptable salt thereof in a form releasable into blood, and can prevent and/or treat restenosis or reocclusion of blood vessels caused by a vascular recanalization operation. The releasable forms of the aforementioned compound or the physiologically acceptable salt thereof into blood are not particularly limited. Examples of the forms include a form wherein the medicament prepared as a sustained release preparation is pasted or applied on the surface of the medical device, as well as coating formation by application on the surface of a medical device or impregnation in the material of a medical device. However, the forms are not limited to these examples. Among the medical devices of the present invention, a preferred example includes an intravascular indwelling stent. The stent contains the compound represented by the formula (I) or the physiologically acceptable salt thereof in a form that enables a sustained release of said substances.

Types of base materials for preparing the stent are not particularly limited. Stainless steel (SUS316, SUS304), Nitinol (Ni—Ti alloy), metallic materials such as tantalum, and polymer materials can be generally used. Biodegradable polymer materials can also be used. As for the polymer materials, types of the materials are not particularly limited so far that they have blood compatibility and are not dissolvable in blood. Methods for producing the stent of the present invention are not particularly limited. For example, when the stent base material consists of a metal, a polymer coating layer comprising the compound represented by the aforementioned formula (I) or the physiologically acceptable salt thereof can be provided on the surface of the stent base material, or when the base material consists of a polymer material, the compound represented by the aforementioned formula (I) or the physiologically acceptable salt thereof may be introduced during molding of the polymer material, or a polymer coating layer containing the compound represented by the aforementioned formula (I) or the physiologically acceptable salt thereof can be provided on the surface of the stent base material.

Types of the polymer materials to form the coating layer are not particularly limited so far that the materials have blood compatibility and are not dissolvable in blood. For example, polyester type elastomers, polyamide type elastomers, polyurethane type elastomers,. (meth)acrylate ester type polymers, polyvinyl acetates, poly(ethylene-vinyl alcohol) copolymers and the like can be used. A polymer material having compliance respondable to expansion of a stent is more desirable.

Each concentration of the compound represented by the aforementioned formula (I) or the physiologically acceptable salt thereof and the aforementioned polymer in a solution for coating can be suitably chosen depending on conditions including, for example, an amount to be eluted (an eluting rate) of the aforementioned active ingredient from the surface of a coated layer and the shape of the stent. The stent is desirably designed so as to have a sustained release property in such a degree that the effectiveness of the medicament of the present invention is maintained for at least a given period of time. It is generally desirable to design the stent so that a local concentration of the active ingredient can be 10 μM to 1 nM.

Methods for producing a stent and means for sustained release are well known and conventionally used by artisans in the fields of stents, artificial organs and the like. For example, as for drug-releasing stents for prevention of restenosis, a review by Kozuma is published (Kozuma, K., Coronary Intervention, Vol. 1, pp. 58-62), and specific drug-releasing stents are described in Catheterization and Cardiovascular Interventions, Vol. 55, pp. 409-417, 2002; New England Journal of Medicine, Vol. 346, pp. 1770-1771 and 1773-1780, 2002; WO02/064065 and the like. The entire disclosures of these publications and the publications cited therein are incorporated by reference in the disclosures of the specification. By referring to these publications, those skilled in the art can readily produce these stents of the present invention. The intravascular balloon catheter and other medical devices for intravascular treatment can also be readily produced by suitably applying these known techniques.

The medicament of the present invention and the medical devices of the present invention may be used in combination. For example, oral or parenteral administration of the medicament of the present invention may be carried out before a vascular recanalization operation, and then vascular recanalization operation may be carried out by using the medical devices of the present invention. Furthermore, oral or parenteral administration of the medicament of the present invention may be carried out intraoperatively and/or postoperatively in addition to the aforementioned administration, if necessary. The medicament of the present invention and preventive and/or therapeutic methods of the present invention can be applied to human, or to mammals other than human including, for example, monkey, dog, pig, rabbit, guinea pig, hamster, rat, and mouse.

### EXAMPLES

### Example 1

### Test for Inhibition of Proliferation of Neointima in a Mouse Model of Femoral Arterial Angiopathy

As an angiopathy model resulting from a vascular recanalization operation, a treatment was carried out in which 0.015 inch spring wires were implanted into both femoral aortas of 20-25 g male C57BL/6 mouse under anesthesia, and placed for 1 minute to extend the arteries. After the operation, the compound represented by the formula (I) was administered 1 mg/kg/day intraperitoneally for 10 days. Blood vessels were extirpated 4 weeks after the operation and histopathologically examined. Influences between the drug administered group and non-administered group on the cross-section ratio of neointima and tunica media (neointima/tunica media) were compared. As a result, the cross-section ratios of the neointima and tunica media were found to be 1.01±0.27:2.01±0.15 for the compound (I) administered group and non-administered group, respectively. Accordingly, it was confirmed that the compound represented by the formula (I) strongly inhibited the proliferation of neointima after the angiopathy, and that the medicament of the present invention was effective for preventive and/or therapeutic treatment of restenosis or reocclusion after a vascular recanalization operation.

### INDUSTRIAL APPLICABILITY

The present invention provides a medicament highly effective for therapeutic and/or preventive treatment of restenosis or reocclusion after a vascular recanalization operation. Furthermore, restenosis or reocclusion after a vascular recanalization operation can be prevented effectively by using the medical devices of the present invention.

What is claimed is:

1. A medicament for therapeutic and/or preventive treatment of restenosis or reocclusion after a vascular recanalization operation, which comprises as an active ingredient a compound represented by the following formula or a physiologically acceptable salt thereof.

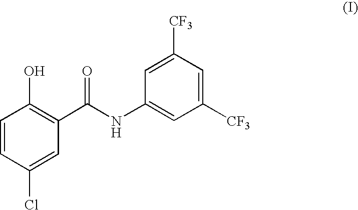

2. A medical device for an intravascular treatment, which contains the compound according to claim 1 or the physiologically acceptable salt thereof in a releasable form.

3. The medical device according to claim 2, which is an intravascular indwelling stent.
